(19) United States
(12) Patent Application Publication
(10) Pub. No.: US 2022/0265226 A1
(43) Pub. Date: Aug. 25, 2022

(54) AN IMAGING METHOD, A SYSTEM AND A RADIOTHERAPY DEVICE BASED ON DUAL-ENERGY CBCT

(71) Applicant: SUZHOU LINATECH INTELLIGENT SCIENCE AND TECHNOLOGY CO., LTD., Suzhou, Jiangsu (CN)

(72) Inventors: Hu Er WEN, Suzhou, Jiangsu (CN); Xuanjia FEI, Suzhou, Jiangsu (CN); Jonathan Yi YAO, Suzhou, Jiangsu (CN)

(21) Appl. No.: 17/630,147

(22) PCT Filed: Nov. 12, 2019
(86) PCT No.: PCT/CN2019/117387

§ 371 Date: Jan. 25, 2022

(30) Foreign Application Priority Data
Oct. 22, 2019 (CN) 201911004192.2

(51) Int. Cl. A61B 6/03; A61B 6/00; A61N 5/10

(52) U.S. Cl. CPC A61B 6/032; A61B 6/4007; A61B 6/4085; A61B 6/4435; A61B 6/482; A61B 6/505; A61B 6/5205; A61B 6/5258; A61N 5/1049; A61N 2005/1061

(57) ABSTRACT

The invention provides an imaging method, a system and a radiotherapy device based on dual-energy CBCT. The method includes: rotating the large gantry by 90°, and obtaining the megavolt projection data from 0° to 90° and the kilovolt projection data from 90° to 180° in the process of rotation; using a predetermined reconstruction algorithm to reconstruct the megavolt projection data and the kilovolt projection data respectively to obtain the megavolt CBCT volume image and the kilovolt CBCT volume image; using the preset algorithm to obtain the corrected kilovolt projection data; using the preset algorithm to obtain corrected megavolt projection data; the corrected kilovolt projection data and the corrected megavolt projection data are used for hybrid reconstruction to obtain CBCT volume image. By using the kilovolt projection image and the megavolt projection image for hybrid reconstruction, CBCT volume image containing both soft tissue information and bone information are obtained.

### TECHNICAL FIELD

The invention relates to the field of medical technology, in particular to an imaging method, a system and a radiotherapy device based on dual-energy CBCT.

### BACKGROUND

Before or during radiotherapy, medical staff often need to verify the positioning of patients to ensure that the positioning of patients on the treatment bed is consistent with that when scanning computed tomography (CT) images used to formulate treatment plans, so that the target area can absorb the planned dose as much as possible and protect normal tissues as much as possible, that is to ensure the implementation of accurate treatment.

In order to meet the needs of physicists and technicians in clinical positioning verification of radiotherapy patients, cone beam computed tomography (CBCT) technology can be used to obtain the three-dimensional volume image of patients in the treatment room, and then conduct three-dimensional registration with the planned CT image to determine the positioning deviation of patients, At this point, the medical staff can correct the patient's positioning according to the positioning deviation.

According to different ray energy levels, CBCT technology can be divided into kilovolt CBCT (KVCBCT) and megavolt CBCT (MVCBCT). Among them, American Varian company and Swedish MEDA company adopt KVCBCT technology, while German Siemens company adopts MVCBCT. In mechanical and electrical aspects, the X-ray beam source of MVCBCT directly adopts the treatment source of the linear accelerator, and the plane of image acquisition board is perpendicular to the X-ray beam axis; The realization of KVCBCT technology requires an additional onboard image system on the traditional megavolt linear accelerator system. The system is composed of a kV X-ray source and a kV image detector installed on two independent mechanical arms respectively. The two mechanical arms are perpendicular to the central axis of the radiation harness of the linac.

When X-rays penetrate the human body, the main roles of X-rays and substances are different according to different energy, resulting in different final imaging quality of CBCT. KV level X-rays mainly have photoelectric effect with material atoms, so KVCBCT can highlight the soft tissue information of human body, but if there are metal products such as metal brackets in human body, KVCBCT will have very serious metal artifacts; MV level X-ray mainly carries out Compton effect with material atoms, so MVCBCT can highlight the bone structure information of human body, but the contrast of human soft tissue is poor.

The existing CBCT system can only realize KVCBCT or MVCBCT. The final three-dimensional volume image can not highlight the soft tissue and bone structure at the same time, and the image has poor anti metal artifact ability, which affects the user's subjective analysis and evaluation.

### SUMMARY

The purpose of the invention is to provide an imaging method, a system and a radiotherapy device based on dual-energy CBCT in view of the shortcomings of the above prior art, so as to solve the problem of acquiring CBCT volume image containing both soft tissue information and bone information, and removing the artifact of high-density substance in the image.

In order to achieve the above purpose, the technical scheme adopted by the invention is as follows:

In the first aspect, the invention provides an imaging method based on d dual-energy CBCT, which is applied to radiotherapy equipment with megavolt imaging subsystem and kilovolt imaging subsystem at the same time, wherein the megavolt imaging subsystem is arranged on the large gantry of radiotherapy equipment, and the kilovolt imaging subsystem is arranged on the independent slip ring of radiotherapy equipment; the rotation center of the independent slip ring is the same as that of the large gantry, and the megavolt image subsystem and kilovolt image subsystem can rotate independently;

the method comprises the following steps:

a, rotating the large gantry by 90°, during the rotation, the megavolt projection data from 0° to 90° is obtained through the megavolt imaging subsystem and the kilovolt projection data from 90° to 180° is obtained through the kilovolt imaging subsystem;

b, a predetermined reconstruction algorithm is used to reconstruct the megavolt projection data and the kilovolt projection data respectively to obtain the megavolt CBCT volume image and the kilovolt CBCT volume image;

c, based on the megavolt CBCT volume image, a preset artifact removal algorithm is used to obtain the corrected kilovolt projection data, the preset artifact removal algorithm is used to remove the artifact in the kilovolt CBCT volume image;

d, based on the kilovolt CBCT volume image, the corrected megavolt projection data is obtained by using the preset soft tissue enhancement algorithm, which is used to enhance the soft tissue image in the megavolt CBCT volume image;

e, the corrected kilovolt projection data and the corrected megavolt projection data are used for hybrid reconstruction to obtain the corrected CBCT volume image.

Alternatively, step c includes:

Step c1: calculating the gradient value of the megavolt CBCT volume image, and obtaining the position information of the high-density substance in the megavolt CBCT volume image according to the gradient value, the high-density substance is a substance whose density is greater than the density of human bone;

Step c2: forward projecting the megavolt CBCT volume image to obtain the megavolt projection data from 90° to 180°, and obtaining the projection position of the high-density substance from the 90° to 180° megavolt projection data according to the position information of the high-density substance in the megavolt CBCT volume image;

Step c3: registering the kilovolt projection data from 90° to 180° and the megavolt projection data from 90° to 180° to obtain the corrected kilovolt projection data.

Alternatively, step c3 includes:

registering the kilovolt projection data from 90° to 180° and the megavolt projection data from 90° to 180° to obtain the high-density substance projection position of the kilovolt projection data from 90° to 180°, the pixel value of the high-density substance projection position area is replaced by the pixel value of the surrounding area by linear interpolation, so as to obtain the corrected kilovolt projection data.

Alternatively, step d includes:

Step d1: forward projecting the kilovolt CBCT volume image to obtain kilovolt projection data from 0° to 90°;

Step d2: normalizing the kilovolt projection data from 0° to 90°, and taking the normalized data value as the weight of each point on the projection plate;

Step d3: using the weight to correct the kilovolt projection data from 0° to 90° to obtain the corrected megavolt projection data.

Optionally, after step e, it also includes:

determining whether the corrected CBCT volume image meets the preset image quality standard;

when the corrected CBCT volume image does not meet the preset image quality standard,

using the predetermined reconstruction algorithm to obtain the corrected megavolt CBCT volume image based on the corrected megavolt projection data, and to obtain the corrected kilovolt CBCT volume image based on the corrected kilovolt projection data;

based on the corrected megavolt CBCT volume image and the corrected kilovolt CBCT volume image, repeating steps c to e until the corrected CBCT volume image meets the preset image quality standard.

In the second aspect, the invention provides a system based on dual-energy CBCT, which is applied to radiotherapy equipment with megavolt imaging subsystem and kilovolt imaging subsystem at the same time, wherein the megavolt imaging subsystem is arranged on the large gantry of radiotherapy equipment, and the kilovolt imaging subsystem is arranged on the independent slip ring of radiotherapy equipment; the rotation center of the independent slip ring is the same as that of the large gantry, and the megavolt imaging subsystem and kilovolt imaging subsystem can rotate relatively independently;

the system comprises:

a projection data acquisition module, for rotating the large gantry by 90°, during the rotation, the megavolt projection data from 0° to 90° is obtained through the megavolt imaging subsystem and the kilovolt projection data for 90° to 180° is obtained through the kilovolt imaging subsystem;

a volume image reconstruction module, for using a predetermined reconstruction algorithm to obtain a megavolt CBCT volume image based on the megavolt projection data and a kilovolt CBCT volume image based on the kilovolt projection data respectively;

a kilovolt projection data correction module, which is used to obtain the corrected kilovolt projection data by using a preset artifact removal algorithm based on the megavolt CBCT volume image, the preset artifact removal algorithm is used to remove the artifact in the kilovolt CBCT volume image;

a megavolt projection data correction module, which is used to obtain the corrected megavolt projection data based on the kilovolt CBCT volume image by using a preset soft tissue enhancement algorithm, and the preset soft tissue enhancement algorithm is used to enhance the soft tissue image in the megavolt CBCT volume image;

a CBCT volume image hybrid reconstruction module, which is used for hybrid reconstruction using the corrected kilovolt projection data and the corrected megavolt projection data to obtain the corrected CBCT volume image.

Optionally, the kilovolt projection data correction module is specifically used for:

calculating the gradient value of the megavolt CBCT volume image, and obtaining the position information of the high-density substance in the megavolt CBCT volume image according to the gradient value, the high-density substance is a substance whose density is greater than the density of human bone;

forward projecting the megavolt CBCT volume image to obtain the megavolt projection data from 90° to 180°, and obtaining the projection position of the high-density substance of the megavolt projection data from 90° to 180° according to the position information of the high-density substance in the megavolt CBCT volume image;

registering the kilovolt projection data from 90° to 180° and the megavolt projection data from 90° to 180° to obtain corrected kilovolt projection data.

Optionally, the kilovolt projection data correction module is specifically used for:

registering the kilovolt projection data from 90° to 180° and the megavolt projection data from 90° to 180° to obtain the high-density substance projection position of the kilovolt projection data from 90° to 180°, the pixel value of the high-density substance projection position area is replaced by the pixel value of the surrounding area by linear interpolation, so as to obtain the corrected kilovolt projection data.

Optionally, a megavolt projection data correction module, specifically for:

forward projecting the kilovolt CBCT volume image to obtain kilovolt projection data from 0° to 90°;

normalizing the kilovolt projection data from 0° to 90°, and taking the normalized data value as the weight of each point on the projection plate;

using the weight to correct the kilovolt projection data from 0° to 90° to obtain the corrected megavolt projection data.

Alternatively, the system also includes a volume image quality judgment and correction module, which is specifically used for:

determining whether the corrected CBCT volume image meets the preset image quality standard;

when the corrected CBCT volume image does not meet the preset image quality standard,

using the predetermined reconstruction algorithm to obtain the corrected megavolt CBCT volume image based on the corrected megavolt projection data, and to obtain the corrected kilovolt CBCT volume image based on the corrected kilovolt projection data;

based on the corrected megavolt CBCT volume image and the corrected kilovolt CBCT volume image, repeatedly run the kilovolt projection data correction module, the megavolt projection data correction module and the CBCT volume image hybrid reconstruction module until the corrected CBCT volume image meets the preset image quality standard.

In a third aspect, the invention provides a radiotherapy device for implementing the dual-energy CBCT based on the imaging method according to the first aspect, or the radiotherapy device includes the dual-energy CBCT based on the imaging system according to the second aspect.

The beneficial effects of the invention include:

the imaging method provided by the invention includes: rotating the gantry by 90°, during the rotation process, obtaining the megavolt projection data from 0° to 90° through the megavolt imaging subsystem and the kilovolt projection data from 90° to 180° through the kilovolt imaging subsystem; using a predetermined reconstruction algorithm to reconstruct the megavolt projection data and the kilovolt projection data respectively to obtain the megavolt CBCT volume image and the kilovolt CBCT volume image; based on the megavolt CBCT volume image, the corrected kilovolt projection data is obtained by using the preset artifact removal algorithm, the preset artifact removal algorithm is used to remove the artifact in the kilovolt CBCT volume image; based on the kilovolt CBCT volume image, the corrected megavolt projection data is obtained by using the preset soft tissue enhancement algorithm, the preset soft tissue enhancement algorithm is used to enhance the soft tissue image in the megavolt CBCT volume image; the corrected kilovolt projection data and the corrected megavolt projection data are used for hybrid reconstruction to obtain the corrected CBCT volume image. CBCT volume image containing both soft tissue information and bone information is obtained by hybrid reconstruction using kilovolt projection image and megavolt projection image, which combines the advantages of clear soft tissue of kilovolt images and weak artifacts of high-density substances (such as metals) of megavolt images, the artifacts of high-density substances in the reconstructed CBCT volume image are removed and the soft tissue information of the image is enhanced.

### BRIEF DESCRIPTION OF THE DRAWINGS

In order to more clearly explain the technical scheme of the embodiment of the invention, the following will briefly introduce the drawings needed to be used in the embodiment. It should be understood that the following drawings only show some embodiments of the invention, so they should not be regarded as limiting the scope. For ordinary technicians in the art, without paying creative labor, Other relevant drawings can also be obtained from these drawings.

FIG. 1 shows a structural diagram of a radiotherapy device provided by an embodiment of the present invention;

FIG. 2 shows a flowchart of an imaging method based on dual-energy CBCT provided by an embodiment of the present invention;

FIG. 3 shows a flowchart of an imaging method based on dual-energy CBCT provided by another embodiment of the present invention;

FIG. 4 shows a structural diagram of a system based on dual-energy CBCT provided by an embodiment of the present invention.

Reference mark: 101—fixed frame; 102—gantry; 103—independent slip ring; 104 megavolt X-ray source; 105 megavolt image detector; 106 kV X-ray source; 107 kV image detector; 108—independent slip ring drive motor.

### DETAILED DESCRIPTION OF THE EMBODIMENTS

The technical scheme in the embodiment of the invention will be clearly and completely described below in combination with the accompanying drawings in the embodiment of the invention. Obviously, the described embodiments are only part of the embodiments of the invention, not all of the embodiments of the invention. Based on the embodiments of the invention, all other embodiments obtained by those skilled in the art without creative work belong to the protection scope of the invention.

The existing CBCT system can only realize KVCBCT or MVCBCT. The final three-dimensional volume image can not highlight the soft tissue and bone structure at the same time, and the image has poor anti metal artifact ability, which affects the user's subjective analysis and evaluation.

In order to solve the above problems, the embodiment of the invention provides a simple and fast CBCT reconstruction method, which can obtain the CBCT volume image containing both soft tissue information and bone information, and can remove the metal artifacts in the image. The technical scheme of the invention includes the following steps: first, establish a radiotherapy equipment with a set of megavolt imaging subsystem and a set of kilovolt imaging subsystem at the same time, wherein the megavolt imaging subsystem is fixed on the gantry of the radiotherapy equipment, the kilovolt imaging subsystem is fixed on an independent slip ring, and the rotation center of the independent slip ring is the same as that of the gantry, the independent slip ring can rotate with the gantry or rotate independently with the gantry; then a hybrid reconstruction algorithm of kilovolt projection image and megavolt projection image is proposed. Combined with the advantages of clear soft tissue of kilovolt image and weak metal artifact of megavolt image, it is used to remove metal artifact in the image and enhance the soft tissue information of the image.

The method provided by the embodiment of the present invention will be described in detail below.

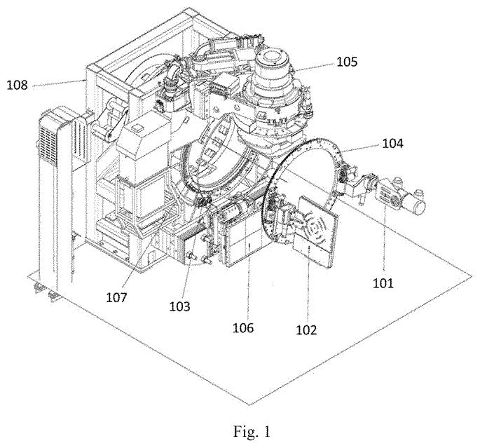

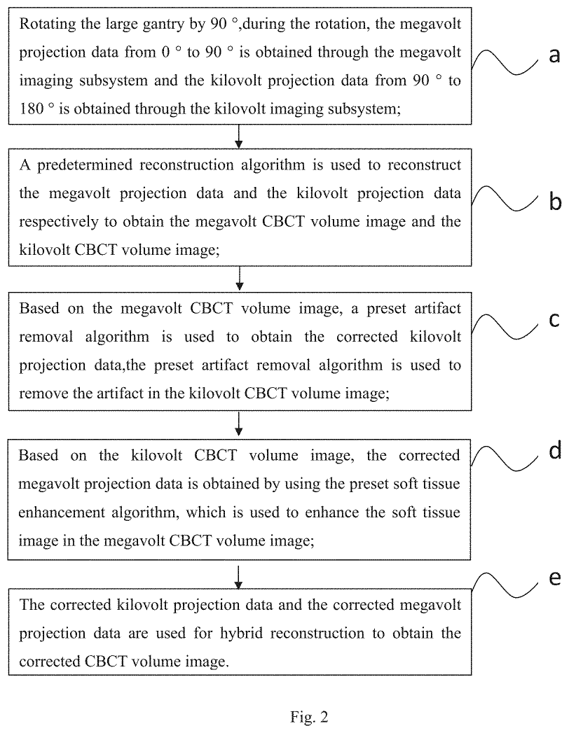

FIG. 1 shows a structural diagram of a radiotherapy device provided by an embodiment of the present invention; FIG. 2 shows a flowchart of an imaging method based on dual-energy CBCT provided by an embodiment of the present invention.

The imaging method based on dual-energy CBCT provided by the embodiment of the invention is applied to the radiotherapy equipment with both megavolt imaging subsystem and kilovolt imaging subsystem. The radiotherapy equipment is shown in FIG. 1, wherein the megavolt imaging subsystem is arranged on the gantry of the radiotherapy equipment, and the kilovolt imaging subsystem is arranged on the independent slip ring of the radiotherapy equipment; The rotation center of the independent slip ring is the same as that of the gantry, and the megavolt image subsystem and kilovolt image subsystem can rotate relatively independently.

Specifically, as shown in FIG. 1, the radiotherapy equipment includes a fixed frame 101, a gantry 102, an independent slip ring 103, a kilovolt (KV) imaging subsystem and a megavolt (MV) imaging subsystem. The gantry 102 is rotatably installed on the fixed frame 101, the megavolt imaging subsystem is fixedly arranged on the gantry 102, and the kilovolt imaging subsystem is fixedly arranged on the independent slip ring 103, the rotation axis of the independent slip ring 103 is the same as that of the gantry 102. The independent slip ring 103 can rotate with the gantry 102 or rotate independently from gantry frame 102. The megavolt image subsystem is used to collect megavolt two-dimensional images and includes a megavolt X-ray source 104 and a megavolt image detector 105, the kilovolt image subsystem is used to collect kilovolt two-dimensional images and includes a kilovolt X-ray source 106 and a kilovolt image detector 107.

When the above radiotherapy equipment is used for treatment, the rotation speed of the independent slip ring 103 and the gantry 102 are controlled respectively through the central controller of the radiotherapy equipment. The gantry 102 drives the MV imaging subsystem to rotate 90°, and the scanning covers the 90° area. The independent slip ring 103 drives the KV imaging subsystem to rotate 90° independently relative to the gantry 102, the scanning coverage is another 90° area that does not overlap with the scanning coverage area of MV imaging subsystem. Among them, the central controller of the radiotherapy equipment controls the gantry 102 to start and rotate 90° according to the specified rotation speed of each circle for 1 minute. At the same time, it controls the independent slip ring 103 to drive the KV imaging subsystem to start and rotate in the same direction with the gantry 102 at the same time, but its rotation speed is faster than the gantry 102. When the gantry 102 completes the 90° rotation stop, After the MV imaging subsystem synchronously scans the 90° area it passes, the independent slip ring 103 also stops rotating, and the KV imaging subsystem just scans the other 90° area that the MV imaging subsystem has not scanned. Therefore, it only takes the time for the gantry 102 to rotate 90°, and the MV imaging subsystem and KV imaging subsystem jointly complete the 180° area scanning, saving 50% scanning time.

The independent slip ring 103 plays an important role, which enables the MV imaging subsystem and KV imaging subsystem to move relatively independently, which can greatly improve the acquisition efficiency of CT image data and MV image data required for pairing learning described below, as well as the cooperative work efficiency of the two subsystems. For example, after the MV imaging subsystem completes the irradiation at a certain angle (including treatment and MV imaging), it leaves this angle to work at other positions. At this time, the KV imaging subsystem can be moved to this angle to complete KV imaging through the independent slip ring 103, which is different from the scheme in the prior art that the relative position between the KV level radiation device and the MV level accelerator is fixed, The technical scheme of the invention has significant advantages.

It should be understood that in the radiotherapy equipment provided by the present invention, the independent slip ring 103 can rotate relatively independently with the gantry 102 as required, and the independent slip ring 103 can also rotate with the gantry 102.

Alternatively, an annular guide rail is fixedly installed on the gantry 102, the annular guide rail shares the same center with the gantry 102, two or more sliders are installed on the annular guide rail, the sliders can rotate freely around the center of the circle along the annular guide rail, and the independent slip ring 103 is installed on the slider, so that the independent slip ring 103 can rotate independently relative to the gantry 102 along the annular guide rail, The rotation axis of the independent slip ring 103 is the same as that of the gantry 102.

A rack or gear is arranged on the outer edge of the independent slip ring 103, and an independent slip ring drive motor 108 is also installed on the gantry 102. The independent slip ring drive motor 108 is connected with the rack or gear on the outer edge of the independent slip ring 103 through gear set or synchronous belt transmission, so that the independent slip ring drive motor 108 can drive the independent slip ring 103 to rotate relative to the gantry 102.

When the independent slip ring drive motor 108 is connected with the rack or gear on the outer edge of the independent slip ring 103 through the synchronous belt drive, in order to prevent the risk caused by the failure of the synchronous belt, two rings of synchronous teeth are arranged on the edge of the independent slip ring 103, and the two rings of synchronous teeth are separated by grooves or flanges. The synchronous belt includes two synchronous belts, the two synchronous belts are respectively matched and connected to two rings of synchronous teeth, and the two synchronous belts are respectively connected to two independent slip ring drive motors 108 respectively arranged on both sides of the gantry 102. One group is used as a standby transmission device to rotate together. When the working synchronous belt fails, the standby synchronous belt will work immediately. Preferably, two independent slip ring drive motors 108 are arranged on both sides of the gantry 102 along the diameter of the gantry 102.

Alternatively, the radiotherapy equipment also includes a safety sensor and a video monitoring device, which are respectively used to sense and monitor the use of the radiotherapy equipment and evaluate the risk of the radiotherapy process to decide whether to stop immediately or continue to complete the treatment plan. The independent slip ring drive motor 108 is electrically connected with the encoder, which is used to control the independent slip ring drive motor 108 and then control the rotation angle of the independent slip ring 103. A holding brake is arranged on the annular guide rail. When the synchronous belt fails, the holding brake is used to stop the rotation of the synchronous slip ring. A plurality of light-emitting elements are uniformly arranged on the annular guide rail, and a detection element is arranged on the independent slip ring 103 corresponding to the starting position of the KV imaging subsystem. The detection element obtains information about at least one of the rotation speed, angular position and rotation direction of the KV imaging subsystem by detecting the light emitted by the light-emitting element. The light-emitting elements are uniformly set according to the preset angle unit, and the wavelength of the light emitted by each light-emitting element is different. The detection element obtains information about at least one of the rotation speed, angular position and rotation direction of the kilovolt imaging subsystem by detecting the wavelength information of the light emitted by the light-emitting element.

As shown in FIG. 2, the dual-energy CBCT based imaging method provided by the embodiment of the invention includes the following steps:

a, rotating the large gantry by 90°, during the rotation, the megavolt projection data from 0° to 90° is obtained through the megavolt imaging subsystem and the kilovolt projection data from 90° to 180° is obtained through the kilovolt imaging subsystem;

b, a predetermined reconstruction algorithm is used to reconstruct the megavolt projection data and the kilovolt projection data respectively to obtain the megavolt CBCT volume image and the kilovolt CBCT volume image;

c, based on the megavolt CBCT volume image, a preset artifact removal algorithm is used to obtain the corrected kilovolt projection data, the preset artifact removal algorithm is used to remove the artifact in the kilovolt CBCT volume image;

d, based on the kilovolt CBCT volume image, the corrected megavolt projection data is obtained by using the preset soft tissue enhancement algorithm, which is used to enhance the soft tissue image in the megavolt CBCT volume image;

e, the corrected kilovolt projection data and the corrected megavolt projection data are used for hybrid reconstruction to obtain the corrected CBCT volume image.

CBCT volume image containing both soft tissue information and bone information is obtained by hybrid reconstruction using kilovolt projection image and megavolt projection image, which combines the advantages of clear soft tissue of kilovolt image and weak artifacts of high-density substances (such as metals) of megavolt image, the artifacts of high-density substances in the reconstructed CBCT volume image are removed and the soft tissue information of the image is enhanced.

Optionally, step c includes:

Step c1: calculating the gradient value of the megavolt CBCT volume image, and obtaining the position information of the high-density substance in the megavolt CBCT volume image according to the gradient value, the high-density substance is a substance whose density is greater than the density of human bone;

Step c2: forward projecting the megavolt CBCT volume image to obtain the megavolt projection data from 90° to 180°, and obtaining the projection position of the high-density substance from the 90° to 180° megavolt projection data according to the position information of the high-density substance in the megavolt CBCT volume image;

Step c3: registering the kilovolt projection data from 90° to 180° and the megavolt projection data from 90° to 180° to obtain the corrected kilovolt projection data.

Alternatively, step c3 includes: registering the kilovolt projection data from 90° to 180° and the megavolt projection data from 90° to 180° to obtain the high-density substance projection position of the kilovolt projection data from 90° to 180°, the pixel value of the high-density substance projection position area is replaced by the pixel value of the surrounding area by linear interpolation, so as to obtain the corrected kilovolt projection data.

Optionally, step d includes:

Step d1: forward projecting the kilovolt CBCT volume image to obtain kilovolt projection data from 0° to 90°;

Step d2: normalizing the kilovolt projection data from 0° to 90°, and taking the normalized data value as the weight of each point on the projection plate;

Step d3: using the weight to correct the kilovolt projection data from 0° to 90° to obtain the corrected megavolt projection data.

Optionally, after step e, it also includes:

determining whether the corrected CBCT volume image meets the preset image quality standard;

when the corrected CBCT volume image does not meet the preset image quality standard,

using the predetermined reconstruction algorithm to obtain the corrected megavolt CBCT volume image based on the corrected megavolt projection data, and to obtain the corrected kilovolt CBCT volume image based on the corrected kilovolt projection data;

based on the corrected megavolt CBCT volume image and the corrected kilovolt CBCT volume image, repeating steps c to e until the corrected CBCT volume image meets the preset image quality standard.

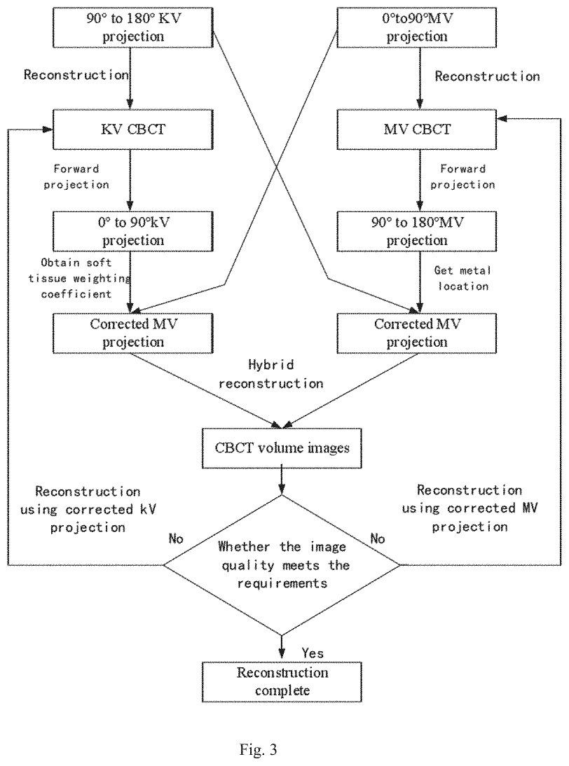

Specifically, referring to FIG. 3, when the CBCT system on the accelerator works, rotate 90° within 15 s to obtain 90° (0° to 90°) MV projection data and 90° (90° to 180°) KV data respectively, reconstruct the projection data respectively to obtain KVCBCT and MVCBCT volume images, and then perform metal artifact correction and soft tissue enhancement; the metal position in the human body can be obtained from the MVCBCT volume image. The MV projection data from 90° to 180° can be obtained by forwarding projection of MVCBCT, and the metal position in the KV projection data can be calculated by registration with the KV projection data from 90° to 180°. The pixel value of the metal area is replaced by the linear interpolation of the surrounding area to obtain the corrected KV projection data; In the KVCBCT volume image, the soft tissue information of the human body can be obtained. By forward projection of KVCBCT, the KV projection data from 0° to 90° can be obtained, and registered with the MV projection data from 0° to 90°, the corresponding positions of the two groups of projection data can be obtained, and the soft tissue area in the KV projection data can be extracted, According to the soft tissue information of KVCBCT, the contrast of the soft tissue area of MV projection data is enhanced to obtain the corrected MV projection data; use the corrected data for reconstruction. If the image quality does not meet the requirements, repeat the above steps with the corrected 90° MV projection data and 90° KV data.

In detail, firstly, the original image acquisition and reconstruction are carried out: the gantry is rotated 90°, and the MV projection data (Proj_{MV}) from 0° to 90° and the KV projection data (Proj_{KV}) from 90° to 180° are obtained respectively; KV projection data and MV projection data are used to reconstruct and then obtain CBCT_{KV} and CBCT_{MV} respectively, and the reconstruction algorithm is (the reconstruction algorithm can be a general algorithm such as FDK or iterative reconstruction).

CBCT_{KV}=f(Proj_{KV})

CBCT_{MV}=f(Proj_{MV})

Then, remove the KV CBCT metal artifact: calculate the gradient value CBCT_{MV}, and obtain the position of high-density substances (such as metals) in the human body according to the gradient extreme value

∇(CBCT_{MV})=0

Perform forward projection on MVCBCT volume image to obtain MV projection data (DRR_{MV})from 90° to 180°, and obtain the metal projection position of DRR_{MV} according to the metal position information in MVCBCT volume image; The Proj_{KV} and Proj_{KV} from 90° to 180° are registered to obtain the metal projection position in Proj_{KV}. The pixel value of the metal area is replaced by the linear interpolation of the surrounding area to obtain a new Proj_{KV}.

Proj′_{KV}=p(Proj_{KV},DRR_{MV})

Use Proj′_{KV} to reconstruct to obtain KVCBCT volume image without metal artifact (Proj′_{KV})

CBCT′_{KV}=f(Proj′_{KV})○

MVCBCT soft tissue enhancement: CBCT′_{KV} is projected forward to obtain the KV projection data (DRR_{KV}) from 0° to 90°, and DRR_{KV} is normalized. The normalized value is the weight ω_{MV} of each point on the plate;

Use the weight ω_{MV} to correct 0° to 90° to obtain the corrected Proj′_{MV}

Proj′_{MV}=p(Proj_{MV},DRR_{KV})=ω_{MV}*Proj_{MV}

Use Proj′_{MV} reconstruction to obtain corrected MVCBCT volume image (CBCT′_{MV})

CBCT′_{MV}=f(Proj′_{MV})○

Dual-energy CBCT reconstruction: Proj′_{KV} and Proj′_{MV} hybrid reconstruction are used to obtain the corrected CBCT.

If the image quality does not meet the requirements, repeat the steps of removing KV CBCT metal artifacts and enhancing MVCBCT soft tissue,

CBCT=f(Proj′_{KV}, Proj′_{MV})○

The imaging method provided by the above embodiment of the invention makes full use of the images of different modes obtained by the accelerator head and KV source for reconstruction, integrates the advantages of CBCT reconstruction volume images of different modes, the obtained volume images contain both soft tissue information and bone information, and solves the problem of metal artifacts caused by foreign bodies such as metal stents in patients. The hybrid reconstruction of different modal data (KVCBCT and MVCBCT) combines the advantages of the two modal volume images through mutual verification and error correction between different modal data.

In addition, the embodiment of the invention provides an imaging system based on dual-energy CBCT, which is applied to radiotherapy equipment with megavolt imaging subsystem and kilovolt imaging subsystem at the same time, wherein the megavolt imaging subsystem is arranged on the gantry of radiotherapy equipment, and the kilovolt imaging subsystem is arranged on the independent slip ring of radiotherapy equipment; the rotation center of the independent slip ring is the same as that of the gantry, and the megavolt imaging subsystem and kilovolt imaging subsystem can rotate relatively independently.

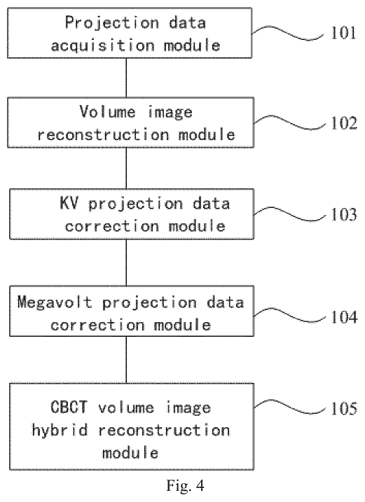

Specifically, the system is used to implement the imaging method based on dual-energy CBCT provided by the above embodiment of the present invention. As shown in FIG. 4, the system comprises:

the projection data acquisition module 101, which is used to rotate the gantry by 90°, during the rotation, the megavolt projection data from 0° to 90° is obtained through the megavolt imaging subsystem and the kilovolt projection data from 90° to 180° is obtained through the kilovolt imaging subsystem;

the volume image reconstruction module 102, which is used to reconstruct megavolt CBCT volume image based on the megavolt projection data and kilovolt CBCT volume image based on the kilovolt projection data by using predetermined reconstruction algorithm respectively;

the kilovolt projection data correction module 103, which is used to obtain the corrected kilovolt projection data based on the megavolt CBCT volume image by using the preset artifact removal algorithm, and the preset artifact removal algorithm is used to remove the artifact in the kilovolt CBCT volume image;

the megavolt projection data correction module 104, which is used to obtain the corrected megavolt projection data based on the kilovolt CBCT volume image by using the preset soft tissue enhancement algorithm, and the preset soft tissue enhancement algorithm is used to enhance the soft tissue image in the megavolt CBCT volume image;

the CBCT volume image hybrid reconstruction module 105, which is used for hybrid reconstruction using the corrected kilovolt projection data and the corrected megavolt projection data to obtain the corrected CBCT volume image.

Optionally, the kilovolt projection data correction module 103 is specifically used to:

calculating the gradient value of the megavolt CBCT volume image, and obtaining the position information of the high-density substance in the megavolt CBCT volume image according to the gradient value, the high-density substance is a substance whose density is greater than the density of human bone;

forward projecting the megavolt CBCT volume image to obtain the megavolt projection data from 90° to 180°, and obtaining the projection position of the high-density substance of the megavolt projection data from 90° to 180° according to the position information of the high-density substance in the megavolt CBCT volume image;

registering the kilovolt projection data from 90° to 180° and the megavolt projection data from 90° to 180° to obtain corrected kilovolt projection data.

Optionally, the kilovolt projection data correction module 103 is specifically used for:

registering the kilovolt projection data from 90° to 180° and the megavolt projection data from 90° to 180° to obtain the high-density substance projection position of the kilovolt projection data from 90° to 180°,the pixel value of the high-density substance projection position area is replaced by the pixel value of the surrounding area by linear interpolation, so as to obtain the corrected kilovolt projection data.

Alternatively, the megavolt projection data correction module 104 is specifically used for:

forward projecting the kilovolt CBCT volume image to obtain kilovolt projection data from 0° to 90°;

normalizing the kilovolt projection data from 0° to 90°, and taking the normalized data value as the weight of each point on the projection plate;

using the weight to correct the kilovolt projection data from 0° to 90° to obtain the corrected megavolt projection data.

Optionally, the system also includes a volume image quality judgment and correction module, which is specifically used for:

determining whether the corrected CBCT volume image meets the preset image quality standard;

when the corrected CBCT volume image does not meet the preset image quality standard,

using the predetermined reconstruction algorithm to obtain the corrected megavolt CBCT volume image based on the corrected megavolt projection data, and to obtain the corrected kilovolt CBCT volume image based on the corrected kilovolt projection data;

based on the corrected megavolt CBCT volume image and the corrected kilovolt CBCT volume image, repeatedly run the kilovolt projection data correction module, the megavolt projection data correction module and the CBCT volume image hybrid reconstruction module until the corrected CBCT volume image meets the preset image quality standard.

In addition, the embodiment of the invention also provides a radiotherapy device for implementing the dual-energy CBCT based on the imaging method provided according to the above embodiment of the invention, or the radiotherapy device includes the dual-energy CBCT based on the system provided according to the above embodiment of the invention.

The above embodiment is only to illustrate the technical concept and characteristics of the invention, and its purpose is to enable ordinary technicians in the art to understand the content of the invention and implement it. It does not limit the protection scope of the invention. All equivalent changes or modifications made according to the spiritual essence of the invention shall be covered by the protection scope of the invention.

1. An imaging method based on dual-energy CBCT is applied to radiotherapy equipment with megavolt imaging subsystem and kilovolt imaging subsystem at the same time, wherein the megavolt imaging subsystem is arranged on the large gantry of the radiotherapy equipment, and the kilovolt imaging subsystem is arranged on the independent slip ring of the radiotherapy equipment; the rotation center of the independent slip ring is the same as that of the large gantry, and the megavolt imaging subsystem and the kilovolt imaging subsystem can rotate relatively independently;
the method comprises the following steps:
a, rotating the large gantry by 90°,during the rotation, the megavolt projection data from 0° to 90° is obtained through the megavolt imaging subsystem and the kilovolt projection data from 90° to 180° is obtained through the kilovolt imaging subsystem;
b, a predetermined reconstruction algorithm is used to reconstruct the megavolt projection data and the kilovolt projection data respectively to obtain the megavolt CBCT volume image and the kilovolt CBCT volume image;
c, based on the megavolt CBCT volume image, a preset artifact removal algorithm is used to obtain the corrected kilovolt projection data, the preset artifact removal algorithm is used to remove the artifact in the kilovolt CBCT volume image;
d, based on the kilovolt CBCT volume image, the corrected megavolt projection data is obtained by using the preset soft tissue enhancement algorithm, which is used to enhance the soft tissue image in the megavolt CBCT volume image;
e, the corrected kilovolt projection data and the corrected megavolt projection data are used for hybrid reconstruction to obtain the corrected CBCT volume image.

2. The method according to claim 1, wherein the step c comprises:
Step c1: calculating the gradient value of the megavolt CBCT volume image, and obtaining the position information of the high-density substance in the megavolt CBCT volume image according to the gradient value, the high-density substance is a substance whose density is greater than the density of human bone;
Step c2: forward projecting the megavolt CBCT volume image to obtain the megavolt projection data from 90° to 180°, and obtaining the projection position of the high-density substance from the 90° to 180° megavolt projection data according to the position information of the high-density substance in the megavolt CBCT volume image;
Step c3: registering the kilovolt projection data from 90° to 180° and the megavolt projection data from 90° to 180° to obtain the corrected kilovolt projection data.

3. The method according to claim 2, wherein the step c3 comprises:
registering the kilovolt projection data from 90° to 180° and the megavolt projection data from 90° to 180° to obtain the high-density substance projection position of the kilovolt projection data from 90° to 180°, the pixel value of the high-density substance projection position area is replaced by the pixel value of the surrounding area by linear interpolation, so as to obtain the corrected kilovolt projection data.

4. The method according to claim 1, wherein the step d comprises:
Step d1: forward projecting the kilovolt CBCT volume image to obtain kilovolt projection data from 0° to 90°;
Step d2: normalizing the kilovolt projection data from 0° to 90°, and taking the normalized data value as the weight of each point on the projection plate;
Step d3: using the weight to correct the kilovolt projection data from 0° to 90° to obtain the corrected megavolt projection data.

5. The method according to claim 1, wherein after step e, it further comprises:
determining whether the corrected CBCT volume image meets the preset image quality standard;
when the corrected CBCT volume image does not meet the preset image quality standard, using the predetermined reconstruction algorithm to obtain the corrected megavolt CBCT volume image based on the corrected megavolt projection data, and to obtain the corrected kilovolt CBCT volume image based on the corrected kilovolt projection data; based on the corrected megavolt CBCT volume image and the corrected kilovolt CBCT volume image, repeating steps c to e until the corrected CBCT volume image meets the preset image quality standard.

6. A system based on dual-energy CBCT is applied to radiotherapy equipment with megavolt imaging subsystem and kilovolt imaging subsystem at the same time, wherein the megavolt imaging subsystem is arranged on the large gantry of the radiotherapy equipment, and the kilovolt imaging subsystem is arranged on the independent slip ring of the radiotherapy equipment; the rotation center of the independent slip ring is the same as that of the large gantry, and the megavolt image subsystem and the kilovolt image subsystem can rotate independently;
the system comprises:
a projection data acquisition module, for rotating the large gantry by 90°, during the rotation, the megavolt projection data from 0° to 90° is obtained through the megavolt imaging subsystem and the kilovolt projection data for 90° to 180° is obtained through the kilovolt imaging subsystem;
a volume image reconstruction module, for using a predetermined reconstruction algorithm respectively to obtain a megavolt CBCT volume image based on the megavolt projection data and a kilovolt CBCT volume image based on the kilovolt projection data;
a kilovolt projection data correction module, which is used to obtain the corrected kilovolt projection data by using a preset artifact removal algorithm based on the megavolt CBCT volume image, the preset artifact removal algorithm is used to remove the artifact in the kilovolt CBCT volume image;
a megavolt projection data correction module, which is used to obtain the corrected megavolt projection data based on the kilovolt CBCT volume image by using a preset soft tissue enhancement algorithm, and the preset soft tissue enhancement algorithm is used to enhance the soft tissue image in the megavolt CBCT volume image;
a CBCT volume image hybrid reconstruction module, which is used for hybrid reconstruction using the corrected kilovolt projection data and the corrected megavolt projection data to obtain the corrected CBCT volume image.

7. The system according to claim 6, wherein the kilovolt projection data correction module is specifically used for:
calculating the gradient value of the megavolt CBCT volume image, and obtaining the position information of the high-density substance in the megavolt CBCT volume image according to the gradient value, the high-density substance is a substance whose density is greater than the density of human bone;
forward projecting the megavolt CBCT volume image to obtain the megavolt projection data from 90° to 180°, and obtaining the projection position of the high-density substance of the megavolt projection data from 90° to 180° according to the position information of the high-density substance in the megavolt CBCT volume image;
registering the kilovolt projection data from 90° to 180° and the megavolt projection data from 90° to 180° to obtain corrected kilovolt projection data.

8. The system according to claim 6, wherein the megavolt projection data correction module is specifically used for:
forward projecting the kilovolt CBCT volume image to obtain kilovolt projection data from 0° to 90°;
normalizing the kilovolt projection data from 0° to 90°, and taking the normalized data value as the weight of each point on the projection plate;
using the weight to correct the kilovolt projection data from 0° to 90° to obtain the corrected megavolt projection data.

9. The system according to claim 6, further comprises a volumetric image quality judgment and correction module, specifically for:
determining whether the corrected CBCT volume image meets the preset image quality standard;
when the corrected CBCT volume image does not meet the preset image quality standard,
using the predetermined reconstruction algorithm to obtain the corrected megavolt CBCT volume image based on the corrected megavolt projection data, and to obtain the corrected kilovolt CBCT volume image based on the corrected kilovolt projection data;
based on the corrected megavolt CBCT volume image and the corrected kilovolt CBCT volume image, repeatedly run the kilovolt projection data correction module, the megavolt projection data correction module and the CBCT volume image hybrid reconstruction module until the corrected CBCT volume image meets the preset image quality standard.

10. A radiotherapy device, which is used to implement the dual-energy CBCT based on the imaging method according to claim 1, or the radiotherapy device includes the dual-energy CBCT based on a system based on dual-energy CBCT is applied to radiotherapy equipment with megavolt imaging subsystem and kilovolt imaging subsystem at the same time, wherein the megavolt imaging subsystem is arranged on the large gantry of the radiotherapy equipment, and the kilovolt imaging subsystem is arranged on the independent slip ring of the radiotherapy equipment the rotation center of the independent slip ring is the same as that of the large gantry, and the megavolt image subsystem and the kilovolt image subsystem can rotate independently;
the system comprises:
a projection data acquisition module, for rotating the large gantry by 90°, during the rotation, the megavolt projection data from 0° to 90° is obtained through the megavolt imaging subsystem and the kilovolt projection data for 90° to 180° is obtained through the kilovolt imaging subsystem;
a volume image reconstruction module, for using a predetermined reconstruction algorithm respectively to obtain a megavolt CBCT volume image based on the megavolt projection data and a kilovolt CBCT volume image based on the kilovolt projection data;
a kilovolt projection data correction module, which is used to obtain the corrected kilovolt projection data by using a preset artifact removal algorithm based on the megavolt CBCT volume image, the preset artifact removal algorithm is used to remove the artifact in the kilovolt CBCT volume image;
a megavolt projection data correction module, which is used to obtain the corrected megavolt projection data based on the kilovolt CBCT volume image by using a preset soft tissue enhancement algorithm, and the preset soft tissue enhancement algorithm is used to enhance the soft tissue image in the megavolt CBCT volume image;
a CBCT volume image hybrid reconstruction module, which is used for hybrid reconstruction using the corrected kilovolt projection data and the corrected megavolt projection data to obtain the corrected CBCT volume image.
